(19) United States
(12) Patent Application Publication
(10) Pub. No.: US 2025/0017974 A1
(43) Pub. Date: Jan. 16, 2025

(54) FECAL TRANSPLANTS FOR REVERSING MOTOR AND GUT SYMPTOMS IN NEURODEVELOPMENTAL DISORDERS

(71) Applicant: California Institute of Technology, Pasadena, CA (US)

(72) Inventors: Sarkis K. Mazmanian, Glendale, CA (US); Matheus de Castro Fonseca, Pasadena, CA (US)

(21) Appl. No.: 18/772,024

(22) Filed: Jul. 12, 2024

Related U.S. Application Data
(60) Provisional application No. 63/526,762, filed on Jul. 14, 2023.

(51) Int. Cl. A61K 35/24; A61K 35/00; A61K 35/741; A61P 25/28

(52) U.S. Cl. CPC A61K 35/24; A61K 35/741; A61P 25/28; A61K 2035/115

(57) ABSTRACT

Disclosed herein are methods that can be used to improve, treat and/or inhibit neurodevelopmental disorders in subjects in need, for example subjects suffering from Parkinson's disease. Some embodiments include administering an effective amount of fecal microbiota to the subject in need thereof, wherein the fecal microbiota is derived from healthy donors who are younger in age as compared to the subjects suffering from Parkinson's disease. Upon treatment, one or more symptoms of neurodevelopmental disorders can be improved in the subject.

### CROSS-REFERENCE TO RELATED APPLICATIONS

The present application claims the benefit of U.S. Provisional Application No. 63/526,762, filed on Jul. 14, 2023, which is hereby incorporated by reference in its entirety.

### FIELD

Some embodiments described herein relate generally to fecal microbiome transplant from a young healthy donor, which can be used to treat neurodevelopmental disorders such as Parkinson's Disease.

### BACKGROUND

The gut microbiota impacts brain development and function as well as complex behaviors such as anxiety, hyperactivity, vocalization, and social interaction (Sudo, 2004; Clarke, 2013; Neufeld, 2011; Crumeyrolle-Arias, 2014). The aging process significantly impacts the composition of gut bacteria, leading to changes in the microbiome that are associated with poorer health and frailty in older individuals (Leite, 2021). Studies conducted on populations of centenarians have revealed a decline in the activity of a healthy microbiome with age. This includes reduced resilience, an increase in pro-inflammatory microbial species, and diminished functionality compared to the microbiomes of younger individuals. Experiments conducted on mice have demonstrated a correlation between the transplantation of microbiomes from young mice into older ones and the improvement of cognitive functions, suggesting potential benefits of younger microbiomes for the aging brain (Boehme, 2021). However, the implications of this research have yet to be mechanistically explored or applied to age-related brain diseases.

### SUMMARY

In accordance with some embodiments described herein, methods for treating, improving and/or delaying a neurodegenerative disorder or a symptom thereof in a subject in need thereof are provided. In some embodiments, the method comprises identifying a subject having a neurodegenerative disorder, or a symptom thereof and administering to the subject a composition comprising an effective amount of fecal microbiota. In some embodiments, the fecal microbiota is derived from a healthy subject. In some embodiments, the neurodegenerative disorder or the symptom thereof of the subject is treated, improved and/or delayed after administering the composition.

In some embodiments, the healthy subject is younger as compared to the subject having the neurodegenerative disorder or the symptom thereof. In some embodiments, the healthy subject is at least 10 years younger than the subject having the neurodegenerative disorder or a symptom thereof. In some embodiments, the healthy subject is 20 years younger than the subject having the neurodegenerative disorder or a symptom thereof.

In some embodiments, the neurodegenerative disorder is a Parkinson's disease synucleinopathy or Huntington's disease.

In some embodiments, the method improves one or more physical impairments in the subject. In some embodiments, the method improves one or more gastrointestinal (GI) functions of the subject. In some embodiments, the one or more GI functions comprise vomiting, dysphagia, bloating, sialorrhea, constipation, or combinations thereof. In some embodiments, the method relieves constipation of the subject. In some embodiments, the method relieves one or more motor deficit symptoms in the subject. In some embodiments, the one or more motor deficit symptoms comprise tremors, muscle rigidity, bradykinesia, impaired gait, or any combination thereof.

In some embodiments, the subject has an abnormal level of aggregation of α-synuclein (αSyn). In some embodiments, administering the composition decreases a rate and/or level of αSyn aggregation in a brain of the subject. In some embodiments, administering the composition decreases a clearance rate and/or level of insoluble αSyn protein aggregate in the brain the subject. In some embodiments, administering the composition decreases a rate and/or level of αSyn aggregation in a brain of the subject, a clearance rate and/or level of insoluble αSyn protein aggregate or a combination thereof.

In some embodiments, the subject suffers from a synucleinopathy. In some embodiments, the synucleinopathy is Parkinson's disease, dementia with Lewy body disease, multiple system atrophy, or any combination thereof. In some embodiments, the synucleinopathy is Parkinson's disease. In some embodiments, the synucleinopathy is a primary or idiopathic parkinsonism, secondary or acquired parkinsonism, hereditary parkinsonism, Parkinson plus syndromes, multiple system degeneration, or any combination thereof.

Some embodiments provided herein relate to methods of treating, improving and/or delaying a neurodegenerative disorder or a symptom thereof in a subject. In some embodiments, the method comprises identifying a subject having the neurodegenerative disorder or a symptom thereof, determining an age of the subject having the neurodegenerative disorder or a symptom thereof and administering to the subject a composition comprising an effective amount of fecal microbiota. In some embodiments, the fecal microbiota is derived from a healthy subject. In some embodiments, the healthy subject is at least 10 years younger than the subject having the neurodegenerative disorder or a symptom thereof. In some embodiments, the healthy subject is 20 years younger than the subject having the neurodegenerative disorder or a symptom thereof.

In some embodiments, the neurodegenerative disorder is a Parkinson's disease synucleinopathy or Huntington's disease. In some embodiments, the method improves one or more gastrointestinal (GI) functions of the subject. In some embodiments, the one or more GI functions comprise vomiting, dysphagia, bloating, sialorrhea, constipation, or combinations thereof. In some embodiments, the method relieves constipation of the subject. In some embodiments, the method relieves one or more motor deficit symptoms in the subject. In some embodiments, the one or more motor deficit symptoms comprise tremors, muscle rigidity, bradykinesia, impaired gait, or any combination thereof.

In some embodiments, the subject has an abnormal level of aggregation of α-synuclein (αSyn). In some embodiments, administering the composition decreases a rate and/or level of αSyn aggregation in a brain of the subject. In some embodiments, administering the composition decreases a clearance rate and/or level of insoluble αSyn protein aggregate in the brain the subject. In some embodiments, administering the composition decreases a rate and/or level of αSyn aggregation in a brain of the subject, a clearance rate and/or level of insoluble αSyn protein aggregate or a combination thereof.

In some embodiments, the composition is formulated for oral administration. In some embodiments, the composition is formulated for intragastric administration. In some embodiments, the composition is a probiotic composition, a nutraceutical composition, a pharmaceutical composition, or any combination thereof.

In some embodiments, the healthy subject is not exposed to antibiotics prior to fecal microbiota collection. In some embodiments, the subject or the subject in need is not receiving an antibiotic treatment.

In some embodiments, the composition comprising an effective amount of fecal microbiota is administered more than once to the subject. In some embodiments, the composition comprising an effective amount of fecal microbiota is administered every day, monthly or weekly.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIGS. 1A-1C depict fecal microbiome transplant protocol and reduction of gastrointestinal symptoms in alpha-synuclein-overexpressing (ASO) recipients. FIG. 1A shows an exemplary schematic of experimental design (w=weeks; ABX=antibiotic treatment) for converting the microbiome of ASO animals. FIG. 1B shows principal component analysis showing β-diversity in ASO 5mo DONORS, ASO 5mo RECIPIENTS BEFORE 5mo ASO fecal microbiota transplantation (FMT), ASO 5mo RECIPIENTS AFTER 5moASO FMT and WT 2mo DONORS (for comparison) as measured by Aitchison distance. FIG. 1C shows principal component analysis showing β-diversity in ASO 5mo DONORS, ASO 5mo RECIPIENTS BEFORE 2moWT FMT, ASO 5mo RECIPIENTS AFTER 2moWT FMT and WT 2mo DONORS as measured by Aitchison distance. Ellipses indicate 95% confidence intervals between groups.

FIGS. 2A-2J depict microbial load in fecal samples. FIGS. 2A and 2B show raw microbial load in fecal samples as measured by qPCR with universal bacterial primers. FIGS. 2C and 2D show the microbial load in fecal samples after antibiotics treatment. FIGS. 2E and 2F show the microbial load in fecal sample after FMTs relative to control mice. FIGS. 2G and 2H show fecal output of these mice at 0, 15, 20, and 60 minute time points. FIGS. 2I and 2J show quantification of fecal output at 60 minutes.

FIGS. 3A-3J depict improving gastrointestinal function in ASO recipients. The following gastrointestinal function metrics were also evaluated such as fecal water content (FIGS. 3A and 3B), gastrointestinal transit time (FIGS. 3C and 3D), colonic bead expulsion (FIGS. 3E and 3F), gastric emptying (FIGS. 3G and 3H) and geometric center (FIGS. 3I and 3J). 5 month-old WT animals that received WT autologous FMT (WT+5moWT FMT); ASO animals that received ASO autologous FMT (ASO+5moASOFMT); ASO animals that received young (2 months old) WT FMT (ASO+2moWTFMT); ASO animals that received FMT from young (2 months old) ASO animals (ASO+2moASOFMT). Data analyzed by two-way ANOVA followed by Tukey's multiple comparisons test. Bars represent mean±SEM. ns, not significant, *p<0.05, **p<0.01; ***p<0.001; ****p<0.0001.

FIGS. 4A-4L depict improving motor symptoms in ASO recipients. FIGS. 4A-4D show animal weights monitored throughout the process of the fecal transplant. N=at least 8 animals per group. The following motor behavior metrics were also evaluated such as beam transversal (FIGS. 4E-H), adhesive removal (FIGS. 41 and 4J), and pole descend (FIGS. 4K and 4L). Data was analyzed by two-way ANOVA followed by Tukey's multiple comparisons test. Bars represent mean±SEM. ns, not significant, *p<0.05, **p<0.01; ***p<0.001; ****p<0.0001

FIGS. 5A-5F depict reduction of αSyn aggregation ASO mice after FMT. FIG. 5A and FIG. 5B show notable aggregation of αSyn in the substantia nigra (SN) and striatum brain region of animals by dot blot. FIG. 5C and FIG. 5D show densitometry for substantia nigra and FIG. 5E and FIG. 5F show densitometry for striatum. WT animals that received WT autologous FMT (WT+5moWT FMT); ASO animals that received ASO autologous FMT (ASO+5moASOFMT); ASO animals that received young (2 months old) WT FMT (ASO+2moWTFMT); ASO animals that received FMT from young (2 months old) ASO animals (ASO+2moASOFMT). N=at least 8 animals per group Data analyzed by two-way ANOVA followed by Tukey's multiple comparisons test. Bars represent mean±SEM. *p<0.05, **p<0.01, ***p<0.001, and ****p<0.0001; ns: not significant.

FIGS. 6A-6G depict the effect of age and microbiome on motor and gastrointestinal dysfunction and aggregation of αSyn. FIGS. 6A and 6B illustrate the motor functions as measured by descent time and time to cross. FIGS. 6C and 6D illustrate the gastrointestinal functions as measured by gastrointestinal transit time and latency bead expulsion. FIG. 6E illustrates αSyn aggregation evaluated by dot-blots and FIG. 6F and FIG. 6G illustrate densitometry quantification of αSyn aggregation dot blots of substantia nigra (SN) and striatum brain regions respectively. Data were tested for normality and analyzed by non-parametric Kruskal-Wallis test followed by Dunn's correction. Bars represent mean±SEM. (ns, not significant, *p<0.05, **p<0.01).

FIGS. 7A-7H depict the effect of FMT on improving motor or gastrointestinal function symptoms. Motor functions as measured by adhesive removal, (FIG. 7A) pole descent, (FIG. 7B) beam crossing (FIG. 7C) and number of errors on beam crossing (FIG. 7D), gastrointestinal functions as measured by fecal output (FIG. 7E), gastrointestinal transit time (FIG. 7F), water content (FIG. 7G) and latency bead expulsion (FIG. 7H). ASO animals were transplanted with fecal material from 2mo WT donors treated with vancomycin (V), erythromycin (E), gentamycin/neomycin (G/N) or ampicillin (A) for 2 weeks. Data analyzed one-way ANOVA followed by Dunnet's multiple comparisons test. Bars represent mean±SEM. ns, not significant, *p<0.05, **p<0.01; ***p<0.001; ****p<0.0001.

FIGS. 8A-8C depict the proteomic analysis following fecal transplant. FIG. 8A shows the gene ontology (GO) of the main biological process altered in the substantia nigra of ASO animals after fecal transplant with WT 2-month microbiota. FIGS. 8B and 8C illustrate the autophagy (FIG. 8B) and lysosomal biogenesis (FIG. 8C) in ASO mice before and after fecal transplant from 2-month-old WT mice.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Without being limited by theory, there has been increased attention on the manipulation of gut microbiota to promote better health, and this has been exemplified by procedures like fecal microbiota transplantation (FMT) (D'Haens, 2019). FMT involves the infusion of a fecal suspension obtained from a healthy donor into the gastrointestinal tract of a patient, aiming to restore a healthy microbiota and treat various diseases. When compared to dietary interventions or probiotics, FMT has been shown to bring about more significant changes in the gut microbiota. It has emerged as a promising therapeutic approach for several gut disorders associated with dysbiosis, including Clostridium difficile infection (CDI) and inflammatory bowel disease (IBD), and has demonstrated positive outcomes in clinical settings (D'Haens, 2019; Mullish, 2018).

Parkinson's disease (PD) has conventionally been described as a neurodegenerative condition that progresses over time and is attributable to the deterioration of dopaminergic neurons in the midbrain's substantia nigra pars compacta (Davie, 2008). The key dogma of PD consists in the aggregation of the protein alpha-synuclein (αSyn) within neurons and the loss of neurons associated with PD results in a condition known as Parkinsonism, which encompasses a range of motor symptoms, including muscle rigidity, slowness of movement, tremors, and difficulties in controlling movement (Spillantini, 1997). However, newer research has demonstrated that individuals with Parkinson's disease who have not yet received drug treatment commonly experience gastrointestinal symptoms such as constipation, nausea, and prolonged intestinal transit time, often years prior to receiving a formal diagnosis of the disease (Adams-Carr, 2016; Martinez-Martin, 2011; Mun, 2016). PD is a condition that is both prevalent and associated with age, as it is rare before the age of 50 and affects around 1% of the global population over the age of 65, increasing to approximately 4-5% for those over the age of 85 (Lau, 2006). Since advancing age remains the most significant risk factor for developing PD, its social and economic impact is likely to continue to increase as populations continue to age (Kanaan, 2011; Driver, 2009).

From the perspective of cell biology and physiology, the aging process of the brain closely aligns with features observed in neurodegenerative conditions like PD. For instance, the concept of “inflammaging,” which refers to the age-related increase in pro-inflammatory immune responses, is a significant risk factor for various diseases that are prevalent among the elderly and frequently cause disability (Santaro, 2018). Age represents the primary risk factor for the development and progression of PD, as it affects numerous cellular processes, including increased brain inflammation, which predisposes individuals to neurodegeneration. Although alterations in gut microbiome composition have consistently been linked to PD, the impact of aging on the PD microbiome remains unknown.

Accordingly, in some embodiments described herein, fecal microbiome transplantation from young healthy subjects into subjects having neurodevelopmental disorders is shown. The method comprises administering a composition comprising, consisting essentially of, or consisting of fecal microbiota from a healthy subject to the subject having a neurodevelopmental disorder or a symptom thereof. The subject can be in need of improvement in neurodevelopmental disorder or a symptom thereof.

### Definitions

Unless defined otherwise, technical and scientific terms used herein have the same meaning as commonly understood by one of ordinary skill in the art to which the present disclosure belongs. Sec, e.g. Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). For purposes of the present disclosure, the following terms are defined below.

The section headings used herein are for organizational purposes only and are not to be construed as limiting the subject matter described.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “subject” is an animal, such as a vertebrate, preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is mouse or rat. In some embodiments, the subject is human.

As used herein, the term “treatment” refers to an intervention (e.g., a clinical intervention) made in response to a disease, disorder or physiological condition manifested by a patient, particularly a patient suffering from a neurodegenerative disease, for example Parkinson's diseases. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments the treatment may reduce, alleviate, or eradicate the symptom(s) of the disease(s). As used herein, the term “prevention” refers to any activity that reduces the burden of the individual later expressing those parkinsonian symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

In some non-limiting embodiments, an effective amount or effective dose of a composition may relate to the amount or dose that provides a significant, measurable, or sufficient therapeutic effect towards the treatment of any one or more of the diseases provided herein, such as a synucleinopathy, Parkinson's disease, Huntington's disease, dementia with Lewy bodies, tauopathy, Alzheimer's disease, progressive supranuclear palsy, corticobasal degeneration, Pick's disease, TDP-43 proteopathy, amyotrophic lateral sclerosis, or any combination thereof. In some embodiments, the effective amount or effective dose of a composition or compound may treat, ameliorate, or prevent the progression of symptoms of any one or more of the diseases provided herein. In some embodiments, the neurodegenerative disease is a synucleinopathy which includes but not limited to a primary or idiopathic parkinsonism, secondary or acquired parkinsonism, hereditary parkinsonism, Parkinson plus syndromes or multiple system degeneration, and any combination thereof. Actual dosage levels of active ingredients in an active composition of the presently disclosed subject matter can be varied so as to administer an amount of the active composition that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In some non-limiting examples, an effective amount or effective dose of a composition may relate to the amount or dose that provides a significant, measurable, or sufficient therapeutic effect towards the treatment of neurodevelopmental disorders, or a symptom thereof, for example Parkinson's Disease. In some embodiments, the effective amount or effective dose of a composition may treat, ameliorate, or prevent the progression or development of Parkinson's Disease, or symptoms thereof. The effective amount or effective dose of the composition can be chosen depending on the age of the patient, for example, if the patient is a child, teen or adult. In some embodiments, a child is around 0-6 years old, teen is around 6-18 years old and an adult is 18-120 years old.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The pharmaceutically acceptable or appropriate carrier may include other compounds known to be beneficial to an impaired situation of the GI tract, (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal based products, milk-based powders, infant formulae, tablets, liquid bacterial suspensions, dried oral supplement, or wet oral supplement.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethane sulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the lungs.

As used herein, the term “nutraceutical” refers to a food stuff (as a fortified food or a dietary supplement) that provides health benefits. Nutraceutical foods are not subject to the same testing and regulations as pharmaceutical drugs.

As used herein, the term “probiotic” refers to live microorganisms, which, when administered in adequate amounts, confer a health benefit on the host. The probiotics in accordance with methods and uses and compositions and kits herein may be available in foods and dietary supplements (for example, but not limited to capsules, tablets, powders, and liquids). Non-limiting examples of foods containing probiotic include dairy products such as yogurt, fermented and unfermented milk, smoothies, butter, cream, hummus, kombucha, salad dressing, miso, tempeh, nutrition bars, and some juices and soy beverages. In some embodiments, the probiotic comprises a single microorganism. In some embodiments, the probiotic comprises a combination of microorganisms. In some embodiments, the probiotic comprises a single composition. In some embodiments, the probiotic comprises two or more compositions, which can be used together, for example administered simultaneously or administered sequentially. It is noted that a probiotic can serve as the “active ingredient” or a composition or compositions for use in administration to a subject. That is, the method, use, and/or composition or compositions (either individually or in the aggregate) can comprise an effective amount of probiotic to improve at least one behavior in a subject. In some embodiments, the probiotic is the sole active ingredient for administration to the subject. In some embodiments, the “sole active ingredient” probiotic for administration to the subject can be provided in a composition or in a method or use that is substantially free of or free of bacteria other than the probiotic, antibiotics, and drugs. Even if the probiotic is the “sole” active ingredient, the composition or composition comprising the probiotic may comprise additional substances (such as buffers, bacterial feedstock, excipients, flavors, and/or food) that do not substantially affect the behavior of the subject but may be useful for the function of the probiotic or its administration.

In some embodiments, the probiotic is comprised in a composition or compositions that are substantially free of bacteria (other than the probiotic) and/or drugs or antibiotics. By “substantially free” or “substantially absent”, it is understood that while a bacteria other than the probiotic, drug, and/or antibiotic may be present in trace amounts, the bacteria other than the probiotic, drug, and/or antibiotic have no appreciable effect on the subject. As used herein “effective amount” of probiotic refers to a quantity sufficient to achieve a clinically significant change in a behavior of a subject.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. By “co-administer” it is meant that a first composition described herein is administered at the same time, just prior to, or just after the administration of a second composition.

As used herein, the term “proteopathy” refers to a disease which is caused by abnormal folding or accumulation of proteins. An abnormal protein may gain a toxic function or lose their normal function. It is possible that misfolded proteins can induce the misfolding of otherwise normally folded proteins, resulting in an amplification of the disease (e.g. prion disease). A proteopathy may be an amyloid proteopathy caused by pathogenic accumulation of protein amyloids. Some non-limiting examples of proteopathies include Alzheimer's disease, cerebral β-amyloid angiopathy, retinal ganglion cell degeneration in glaucoma, Parkinson's disease, dementia with Lewy bodies, multiple system atrophy, synucleinopathy, Pick's disease, corticobasal degeneration, tauopathy, progressive supranuclear palsy, TDP-43 proteopathy, amyotrophic lateral sclerosis, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, SAA amyloidosis, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, TTR amyloidosis, cardiac amyloidosis, cardiac atrial amyloidosis, uromodulin-associated kidney disease, IAPP amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), pituitary prolactinoma, insulin amyloidosis, corneal lactoferrin amyloidosis, pulmonary alveolar proteinosis, seminal vesicle amyloid, cutaneous lichen amyloidosis, Mallory bodies, odontogenic (Pindborg) tumor amyloid, cancer, aging promoted by amyloid aggregation, or any disease caused by the misfolding or aggregation of proteins, or otherwise known by a person skilled in the art. The term “proteinopathy” may be used interchangeably with “proteopathy” as understood in the art.

### Exemplary Methods of Use

In some embodiments, a method of treating, improving and/or delaying a neurodegenerative disorder or a symptom thereof in a subject in need thereof is disclosed. In some embodiments, the method comprises: identifying a subject having a neurodegenerative disorder, or a symptom thereof and administering to the subject a composition comprising an effective amount of fecal microbiota. In some embodiments, the fecal microbiota is derived from a healthy subject. In some embodiments, the neurodegenerative disorder or a symptom thereof of the subject is treated, improved and/or delayed after administering the composition comprising an effective amount of fecal microbiota.

In some embodiments, the administration of a composition comprising an effective amount of fecal microbiota treats, improves and/or delays the development of neurodegenerative disorder, or a symptom thereof in the subject by about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the development of neurodegenerative disorder, or a symptom thereof in the subject prior to administration of the composition comprising an effective amount of fecal microbiota. In some embodiments, the neurodegenerative disorder is selected from one or more of Parkinson's disease, Alzheimer's disease, Huntington's disease, cerebral β-amyloid angiopathy, retinal ganglion cell degeneration in glaucoma, dementia with Lewy bodies, multiple system atrophy and synucleinopathy. In some embodiments, the neurodegenerative disorder is Parkinson's disease. Non-limiting examples of neurodegenerative disorders include a primary or idiopathic parkinsonism, secondary or acquired parkinsonism, hereditary parkinsonism, Parkinson plus syndromes or multiple system degeneration, and any combination thereof.

In some embodiments, the one or more symptoms comprise one or more of physical impairments, impaired motor deficits and/or impaired gastrointestinal (GI) functions. In some embodiments, motor deficits include but not limited to tremors, muscle rigidity, bradykinesia, impaired gait or any combination thereof. In some embodiments, GI functions include but not limited to vomiting, dysphagia, bloating, sialorrhea, constipation or any combination thereof. In some embodiments, the administration of a composition comprising an effective amount of fecal microbiota recovers impaired GI functions in the subject in need by about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the GI functions in the subject prior to administration of fecal microbiota. In some embodiments, the administration of a composition comprising an effective amount of fecal microbiota improves motor functions or deficits in the subject in need by about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the motor functions or deficits in the subject prior to administration of fecal microbiota. For example, in some embodiments, administration of an effective amount of fecal microbiota reduces the development of neurodevelopmental disorders, GI dysfunction and motor deficits than expected by between about 1-100, 1-75, 1-50, 1-25, 1-10, 1-5, 5-100, 5-75, 5-50, 5-25, 5-10, 10-100, 10-75, 10-50, 10-25, 25-100, 25-75, 25-50, or 50-100%, as compared to the development of neurodevelopmental disorders, GI dysfunction and motor deficits in the subject prior to administration of an effective amount of fecal microbiota. In some embodiments, the reduction in the development of neurodevelopmental disorders or associated symptoms thereof means, that the neurodevelopmental disorder or one or more corresponding symptoms do not increase in the subject after the administration of an effective amount of fecal microbiota as compared to the neurodevelopmental disorders or one or more corresponding symptoms prior to administration of an effective amount of fecal microbiota.

In some embodiments, the administration of an effective amount of fecal microbiota to a subject in need thereof, converts the microbiome population of the subject in need closer to the microbiome population of the healthy subject. In some embodiments, the administration of an effective amount of fecal microbiota to a subject in need thereof, converts the microbiome population of the subject in need by about 1, 2, 3, 4, 5, 6, 7, 8, 9, 10, 20, 25, 30, 40, 50, 60, 70, 75, 80, 90, or 100%, or by an amount in a range that is defined by any two of the preceding numbers closer to the microbiome population of the healthy subject.

In some embodiments, the subject has been diagnosed with neurodevelopmental disorders or symptoms thereof. In some embodiments, the subject has been diagnosed with a neurodevelopmental disorder or exhibits one or more symptoms thereof. In some embodiments, the subject is at a predisposed risk of developing a neurodevelopmental disorder or associated symptoms thereof. In some embodiments, the subject has a neurodevelopmental disorder or associated symptoms thereof from birth. In some embodiments, the subject develops or developed a neurodevelopmental disorder or associated symptoms thereof during childhood. In some embodiments, the subject does not exhibit any symptoms of a neurodevelopmental disorder. In some embodiments, the neurodevelopmental disorder is synucleinopathy, for example, Parkinson's disease, dementia with Lewy body disease, multiple system atrophy, or any combination thereof. In some embodiments, the subject in need is a subject suffering from or at a risk of developing Parkinson's disease. In some embodiments, the neurodevelopmental disorder is a primary or idiopathic parkinsonism, secondary or acquired parkinsonism, hereditary parkinsonism, Parkinson plus syndromes or multiple system degeneration, and any combination thereof. In some embodiments, the subject in need is identified by a blood test, an ultrasound, an MRI, and CT scan. One of ordinary skill in the art would appreciate that the attending physician would know how to identify a subject in need of treatment disclosed herein.

In some embodiments, the fecal microbiota is derived from a healthy subject. In some embodiments, the healthy subject does not have a neurodevelopmental disorder, or one or more associated symptoms. In some embodiments, the healthy subject does not have Parkinson's Disease or one or more associated symptoms. In some embodiments, the healthy subject is not genetically predisposed to developing a neurodevelopmental disorder, or one or more associated symptoms. In some embodiments, the healthy subject is younger than the subject with a neurodegenerative disorder or a symptom thereof. In some embodiments, the healthy subject is at least 0, 1, 2, 3, 4, 5, 6, 7, 8, 9, 10, 11, 12, 15, 20, 30, 40, 50, 60, 70, 80, 90, 100 months, or any number of months between 1-100 months younger than the subject with a neurodegenerative disorder or a symptom thereof. In some embodiments, the healthy subject is at least 1, 2, 3, 4, 5, 6, 7, 8, 9, 10, 20, 30, 40, 50, 60, 70, 80, 90, 100 years, or any number of years between 1-100 years younger than the subject with a neurodegenerative disorder or a symptom thereof. In some embodiments, the subject is at least 10 years younger than the subject having the neurodegenerative disorder or a symptom thereof. In some embodiments, the subject is at least 20 years younger than the subject having the neurodegenerative disorder or a symptom thereof. In some embodiments, the subject is at least 30 years younger than the subject having the neurodegenerative disorder or a symptom thereof. In some embodiments, the subject is at least 40 years younger than the subject having the neurodegenerative disorder or a symptom thereof. In some embodiments, the subject is at least 50 years younger than the subject having the neurodegenerative disorder or a symptom thereof.

In some embodiments, the healthy subject is not exposed to antibiotics prior to fecal microbiota collection. In some embodiments, the healthy subject is not exposed to antibiotics 1, 2, 3, 4, 5 months or any number of months between 1-5 months prior to donating or prior to collection of fecal microbiota. In some embodiments, the healthy subject is not exposed to antibiotics for at least three months prior to donating or prior to collection of fecal microbiota.

In some embodiments, the composition comprising an effective amount of fecal microbiota is administered more than once to the subject in need. In some embodiments, the composition comprising an effective amount of fecal microbiota is administered at least once, twice, thrice every day. In some embodiments, the composition comprising an effective amount of fecal microbiota is administered at least once a week, twice every week, thrice every week, four times every week, five times every week, six times every week or seven times every week. In some embodiments, the composition comprising an effective amount of fecal microbiota is administered once every month or once every 6 months. In some embodiments, the composition comprising an effective amount of fecal microbiota is administered for up to about 1, 2, 3, 4, 5, 6, 7, 8, 9, 10, 11, 12, 13, 14, 15, 16, 17, 18, 19 or 20 weeks, or for an amount of time that is in a range defined by any two of the preceding values. For example, in some embodiments, the an effective amount of fecal microbiota is administered for between about 1 and 20, 1 and 10, 1 and 8, 1 and 6, 1 and 4, 1 and 2, 1 and 14, 2 and 20, 2 and 10, 2 and 8, 2 and 6, 2 and 4, 4 and 12, 4 and 10, 4 and 8, 4 and 6, 6 and 12, 6 and 10, 6 and 8, 8 and 12, 8 and 10, or 10 and 20 weeks. In some embodiments, an effective amount of fecal microbiota is administered for longer than 20 weeks. In some embodiments, an effective amount of fecal microbiota is administered on an ongoing basis. In some embodiments, the composition is administered to the subject until an improvement in one or more associated symptoms described herein are observed. Optionally, the composition is administered to the subject after an improvement in one or more associated symptoms is observed, for example to solidify or maintain the improved symptoms.

In some embodiments, administration of an effective amount of fecal microbiota inhibits or decreases the clearance rate and/or level of insoluble αSyn protein aggregate in the brain of the subject with neurodevelopmental disorder or one or more symptoms thereof by about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the clearance rate and/or level of insoluble αSyn protein aggregate in the brain of the subject with neurodevelopmental disorder or one or more symptoms thereof prior to administration of an effective amount of fecal microbiota. In some embodiments, the administration of an effective amount of fecal microbiota inhibits or decreases the clearance rate and/or level of insoluble αSyn protein aggregate in the substantia nigra and striatum regions of the brain.

In some embodiments, administration of an effective amount of fecal microbiota regulates the proteins involved in autophagy induction and/or lysosomal biogenesis. In some embodiments, administration of an effective amount of fecal microbiota upregulates the proteins involved in autophagy induction and/or lysosomal biogenesis about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the proteins involved in autophagy induction and/or lysosomal biogenesis in the subject prior to administration of an effective amount of fecal microbiota. In some embodiments, administration of an effective amount of fecal microbiota downregulates the proteins involved in autophagy induction and/or lysosomal biogenesis about 1, 2, 3, 4, 5, 6, 7, 8, 9, 10, 20, 25, 30, 40, 50, 60, 70, 75, 80, 90, or 100%, or by an amount in a range that is defined by any two of the preceding numbers, as compared to the proteins involved in autophagy induction and/or lysosomal biogenesis in the subject prior to administration of an effective amount of fecal microbiota.

As applied to any of the methods of treatment disclosed herein, in some embodiments, the effective amount of fecal microbiota is administered enterally, orally, intranasally, parenterally, intracranially, subcutaneously, intragastrically, intramuscularly, intradermally, or intravenously, or any combination thereof. In some embodiments, the effective amount of fecal microbiota is administered orally. In some embodiments, the effective amount of fecal microbiota is administered intragastrically.

Various criteria can be used to determine the inclusion and/or exclusion of a particular subject in the reference or healthy population, including age of the subject (e.g. the reference subject is younger than the subject in need of treatment) and gender of the subject (e.g. the reference subject can be the same gender or different gender as the subject in need of treatment).

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Experimental Material and Methods

### Fecal Output

Animals were removed from the rack and transferred to the experimental room for 30 min for habituation. Animals were single housed on a sterile empty cage with no bedding for 60 min. Number of fecal pellets produced in 60 min were counted and plotted.

### Fecal Water Content

Each animal was placed into a sterile tricornered beaker until one fecal pellet was produced. Fecal pellets were immediately collected into a 1.5 mL centrifuge tube and weighed (wet content—A) and placed in a benchtop oven at 65° C. overnight. Fecal pellets were weighed again (dry content—B) and total water content was calculated as follows: 100−(B*100/A).

### Gastrointestinal Transit Time

A 6% (w/v) solution of carmine red (Sigma-Aldrich, St. Louis, MO) containing 0.5% methylcellulose (Sigma-Aldrich) was prepared by dissolution and autoclaving before administration. Unfasted mice received an oral gavage with 150 μL of the carmine red solution. Animals were placed back in the original cage and left resting for 150 min. After this period, mice were housed individually without bedding and monitored until the first red fecal pellet was expelled.

### Bead Expulsion Assay

In brief, animals were anesthetized with isoflurane, and a 2 mm glass bead coated with 10% glycerol was gently inserted 2 cm into the rectum. Subsequently, animals were placed in an empty cage and monitored until they expelled the glass bead. If the bead could not be inserted fully due to the presence of a fecal pellet, or if it was immediately expelled with a fecal pellet, the procedure was repeated after the pellet was expelled. The time elapsed between insertion and expulsion of the glass bead was recorded as a measure of colon motility.

### Beam Traversal

A 1-meter-long plexiglass beam (Stark's Plastics, Forest Park, OH) constructed of four segments of 0.25 m length was used. This beam consisted of four individual segments, each with a width decreasing by 1 cm increments along its length (3.5 cm, 2.5 cm, 1.5 cm, and 0.5 cm). The widest segment acted as a loading platform for the animals and the narrowest end placed into the home cage. Animals had one day of training to traverse the length of the beam before testing. On training day mice were encouraged to cross the beam in three successive trials. On a fourth last training trial, animals had no assistance to encourage forward movement and stability on the beam. Timing began when the animals placed their forelimbs onto the 2.5 cm segment and ended when one forelimb reached the home cage. The time taken to cross, and errors were evaluated.

### Pole Descent

The time it took for mice to descend a 24-inch pole wrapped in mesh liner was recorded. The pole was placed in a new sterile clean cage, and they were trained for one day before being tested on the following day. On training day, four trials were conducted: in trial 1, mice were gently placed head down on the pole one-third of the distance from the base; in trial 2, they were placed head down two-thirds of the distance from the base; in trial 3, they were placed head down at the top of the pole; and in trial 4 the animals were placed head up at the top of the pole. On the testing day, mice were placed at the top of the pole for one trial. The timer was stopped once all the limbs touched the base of the pole

### Adhesive Removal

Quarter inch round adhesive labels (Avery, Glendale, CA) were placed on the nasal bridge between the nostrils and forehead. Animals were placed into a new clean empty sterile cage and timed to completely remove the sticker. Animals were recorded over one single trial to avoid excessive stress due to manipulation.

### Dot-Blot Assay

Substantia nigra and striatum were dissected on ice and stored at −80° C. until used. Proteins were extracted using RIPA buffer (Merck) supplemented with Protease and phosphatase inhibitor. Samples were homogenized for 90 s using a tissue homogenizer and were placed directly on ice for 10-15 min following homogenization. Lysates were centrifuged at 13,500 rpm for 10 min and supernatants were collected and stored at −80° C. for later use. Samples were dosed using the BCA method.

Protein samples were normalized to equal concentrations between 1 μg/μL in MilliQ H20 and 1 μL of each sample was carefully spotted onto dry nitrocellulose (0.45 μm pores) membrane. The moisture was allowed to dry entirely. The membrane was blocked in 5% skim milk in TBST (1× Tris-Buffered Saline, 0.1% Tween® 20 Detergent) for 1 hour. Anti-aggregated αSyn antibody was applied in 5% skim milk in TBST (1:1000 for Abcam fibril aggregate specific αSyn antibody: MJFR 14-6-4-2) and incubated at room temperature for 1-2 hrs, or overnight at 4° C. After incubation, it was washed 3× with TBST and incubated with anti-rabbit IgG at 1:1000 for 1-2 hrs in TBST. This process was followed by a wash 3× with TBST and developed with Biorad Chemiluminescent substrate.

### Mass Spectrometry-Based Proteomics

Samples were analyzed using Eclipse mass spectrometer coupled to Vanquish Neo. Peptides were separated on an Aurora UHPLC Column (60 cm×75 μm, 1.7 μm C18, AUR3-60075C18-TS, Ion Opticks) with a flow rate of 0.3 μL/min for a total duration of 2 hour and ionized at 1.8 kV in the positive ion mode. Raw data files were searched against the Uniprot mouse database (UP000000589) using the Proteome Discoverer (PD) 2.5 software based on the Sequest HT algorithm. The search settings were as follows: dynamic modifications: oxidation/+15.995 Da (M), protein N-terminal acetylation/+42.011 Da, GG/+114.043 Da (K, S, T) and protein N-terminal GG/+114.043 Da for ubiquitylation; fixed modification: carbamidomethylation/+57.021 Da (C); precursor mass tolerance: 10 ppm; fragment mass tolerance: 0.6 Da. The maximum false peptide discovery rate was specified as 0.01 using the Percolator Node.

### Example 1. Fecal Microbiome Transplant (FMT) from Wild-Type (WT) Donors Recovers Gastrointestinal Symptoms (GI) in ASO Recipients

This example demonstrates that FMT recovers GI dysfunction in ASO recipients after 4 weeks of treatment.

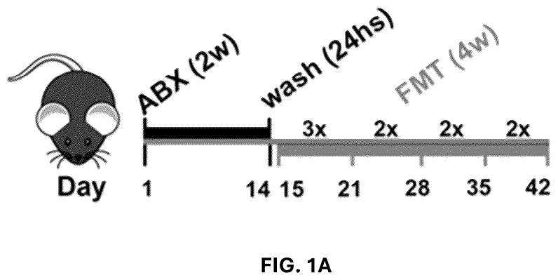

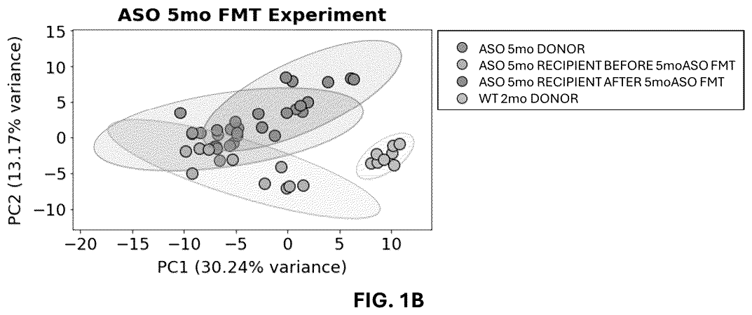

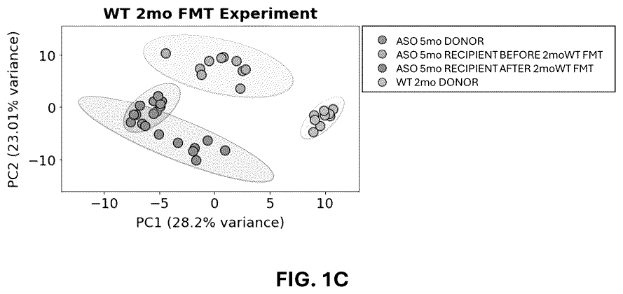

The Thy1-αSyn (alpha-synuclein-overexpressing [ASO]) mouse displays progressive deficits in fine and gross motor function, as well as gut motility defects. Evidence has linked unregulated αSyn expression in humans to a higher risk of PD, providing an epidemiological foundation for the Thy1-αSyn mouse model. To test whether replacing the microbiome of aged mice with young microbiomes by FMT will slow or halt the progression of age-related outcomes such as PD, fecal transplants from young, healthy 2-month-old wild-type (WT) donors, but not 2-month-old ASO donors, into 5-month-old ASO recipient mice were performed. To perform the fecal microbiome transplant, gut microbiota depletion with oral antibiotics (ABX) was performed by administration of ampicillin as sodium salt (1 g ampicillin/L, Patterson Veterinary), vancomycin as hydrochloride salt (0.5 g vancomycin/L, Almaject Inc.), neomycin sulfate (0.5 g/L, Fisher Scientific), gentamycin sulfate (0.1 g/L, Phoenix™), and erythromycin (0.01 g/L, Sigma-Aldrich) to 5-month-old ASO mice for a period of 2 weeks. Drinking ABX was replaced weekly. For the transplants, fecal samples were collected from experimental wild-type mice at 2, 5, and 12 months of age, weighed, and resuspended in a 10-fold volume of sterile filtered (0.22 μm) reduced phosphate buffered saline (PBS) containing 1.5% (w/v) sodium bicarbonate under anaerobic conditions. The sample was mashed with a pipette tip to create a fecal slurry and let to decant. The bacterial supernatant was collected and 150 μL was administered by intragastric gavage to recipient 5-month-old ASO mice that were removed from ABX treatment 24 hrs in advance. This procedure occurred once-daily for three days following antibiotic removal in the first week, and once-daily twice a week for the following next 3 weeks, totaling a period of 4 weeks. Mice were rehoused in a new sterile cage on the first day receiving FMT. Bedding from the donors were also added to the cage to improve bacterial colonization and engraftment. Schematic of experimental design is shown in FIG. 1A. The difference in β-diversity between the ASO 5-month donor mice (ASO 5mo DONORS), ASO 5-month recipient mice before FMT from ASO 5mo FMT (ASO 5mo RECIPIENTS BEFORE 5mo ASO FMT), ASO 5-month recipients after 5-month ASO FMT and WT 2-month donors are shown in FIG. 1B by principal component analysis as measured by Aitchinson distance. Principal component analysis showing β-diversity in ASO 5-month donors, ASO 5-month recipients before 2 month WT FMT, ASO 5-month recipients after 2-month WT FMT and WT 2-month donors as measured by Aitchison distance is illustrated in FIG. 1C. These results illustrate that fecal microbiome transplant is effective in converting the microbiome from ASO animals to closer to that of 2-month-old WT animals.

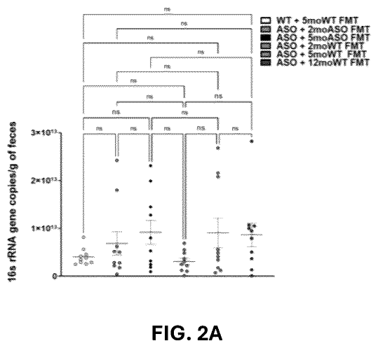

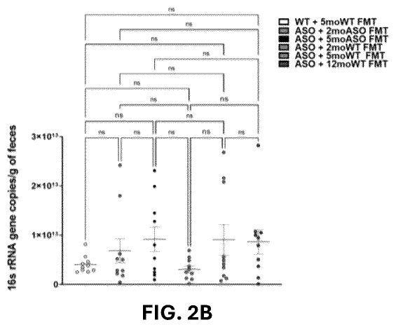

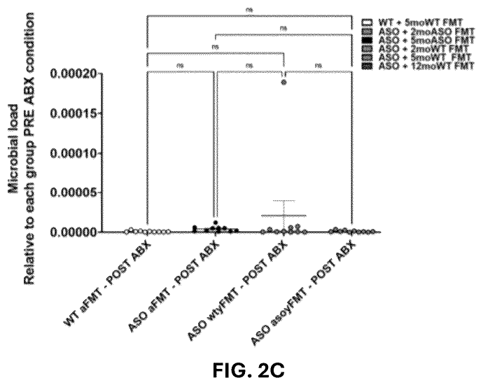

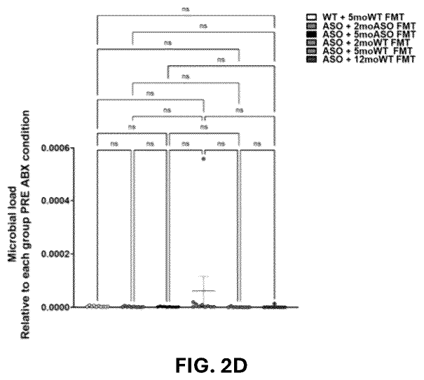

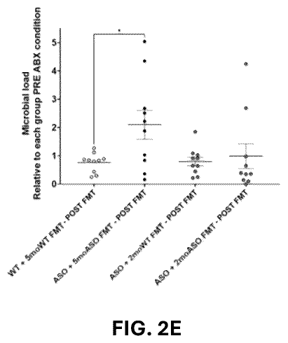

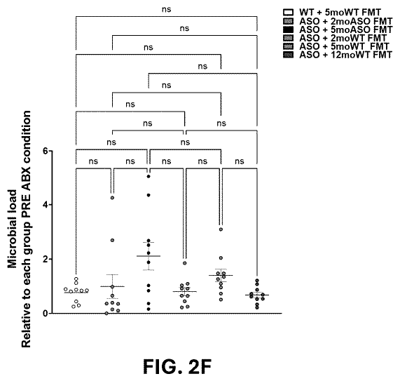

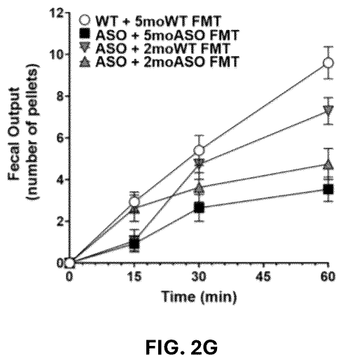

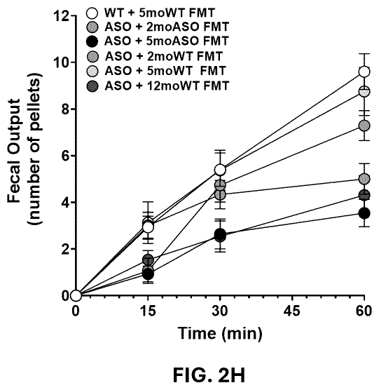

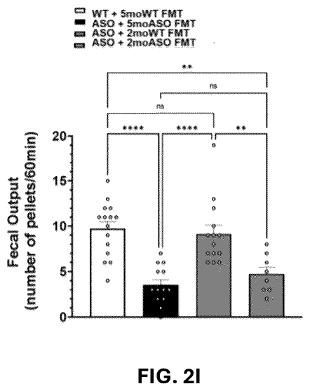

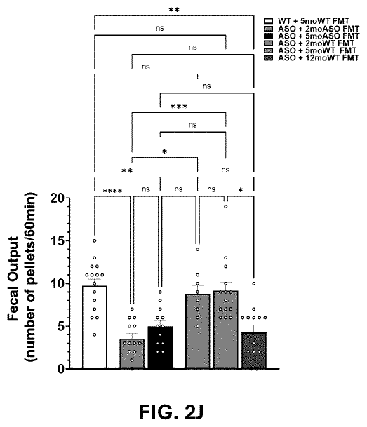

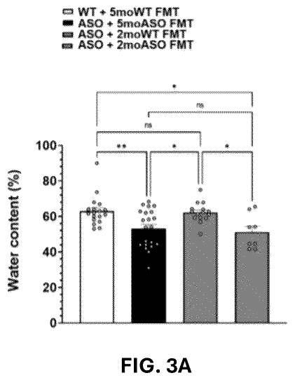

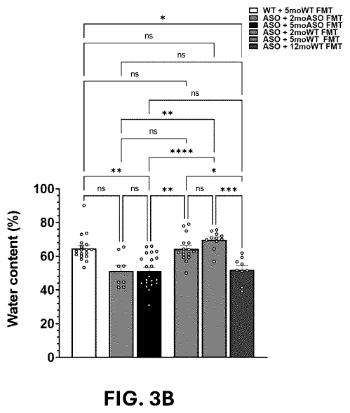

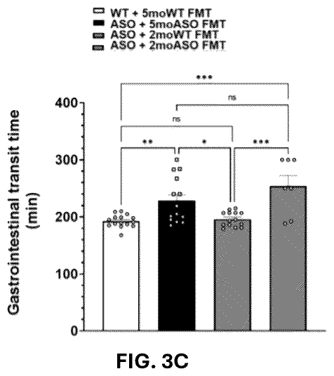

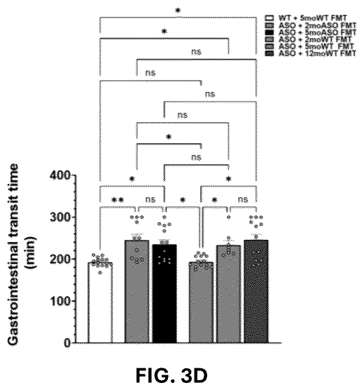

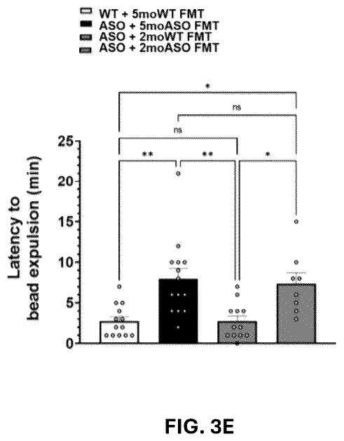

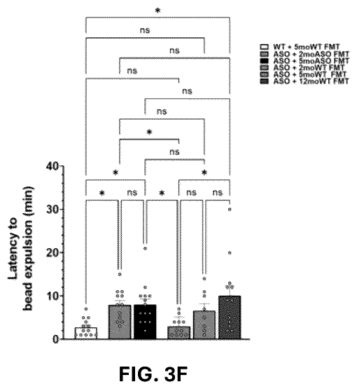

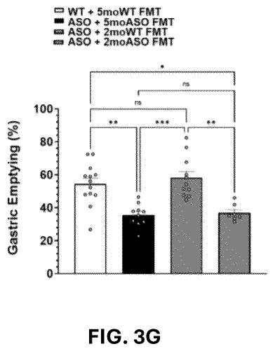

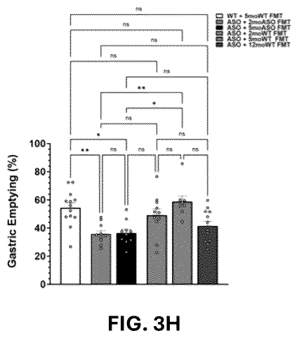

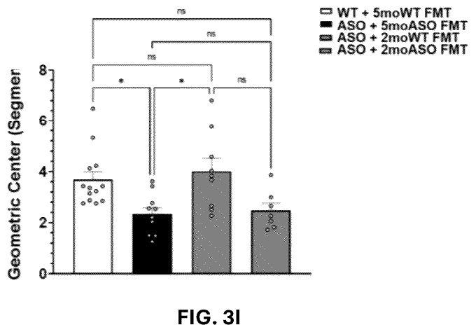

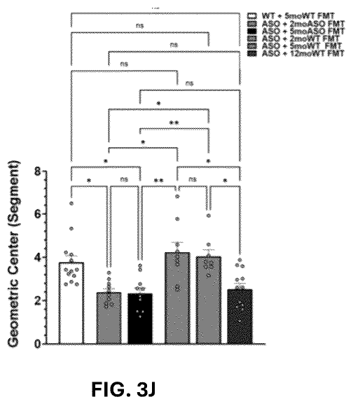

Next, the effect of FMT on improving GI dysfunction in ASO animals was tested. For this analysis, FMT from 2 months old, 5 months old and 12 months old WT mice and 2 months old and 5 months old ASO mice were transplanted into either 5-month old WT or 5-month old ASO mice as shown in FIGS. 2A-2J and 3A-3J. Raw microbial load was measured in fecal samples of mice prior to antibody treatment, after antibiotic treatment and after FMT by measuring 16sRNA gene copies per gram of fecal material. FIG. 2A-2F represent the raw microbial load in fecal samples as measured by qPCR with universal bacterial primers (FIGS. 2A and 2B), in fecal samples after antibiotic treatment (FIGS. 2C and 2D) and after FMTs relative to control mice (FIGS. 2E and 2D). Fecal output of these mice at 0, 15, 20, and 60-minute time points are shown in FIGS. 2G and 2H and quantification of fecal output at 60 minutes at is illustrated in FIGS. 2I and 2J. GI function, as measured by the fecal output of the mice significantly improved in ASO mice treated with FMT from young WT mice as shown in FIGS. 2G-2J, as compared to ASO mice treated with FMT from young ASO mice. These observations were further supported by measuring various gastrointestinal function metrics such as fecal water content (FIGS. 3A and 3B), gastrointestinal transit time (FIGS. 3C and 3D), colonic bead expulsion (FIGS. 3E and 3F), gastric emptying (FIGS. 3G and 3H) and geometric center (FIGS. 3I and 3J) in ASO mice treated with FMT from young WT or ASO mice. These data illustrate that fecal microbiome transplant from WT donors, specifically young WT donors recovered gastrointestinal symptoms in ASO recipients.

### Example 2. Fecal Microbiome Transplant from Young WT Donors to 5-Month-Old ASO Animals Improves Motor Symptoms of ASO Animals after 4 Weeks of Treatment

This example demonstrates that FMT recovers motor dysfunction in ASO recipients.

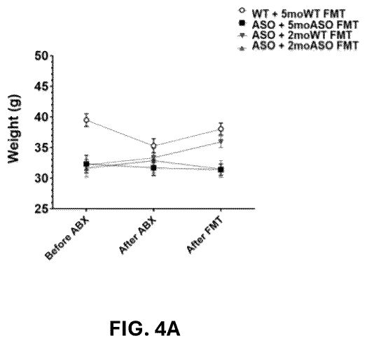

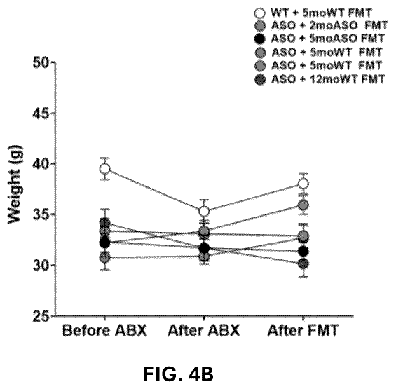

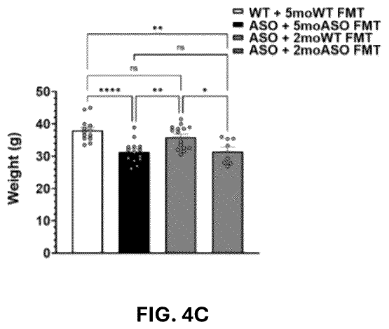

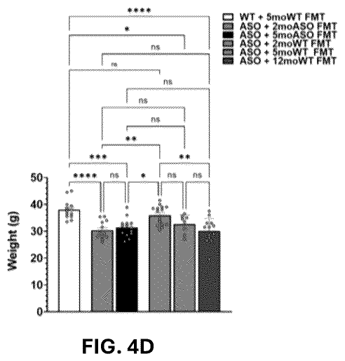

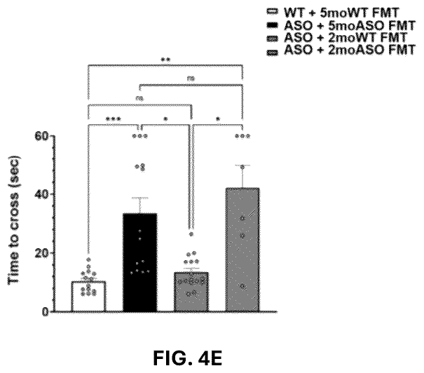

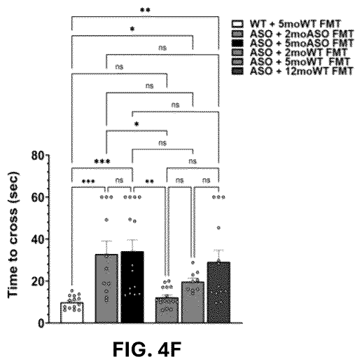

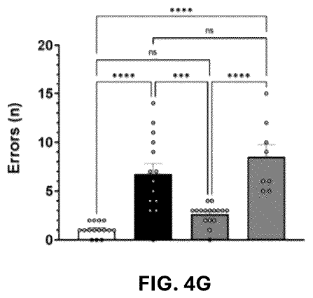

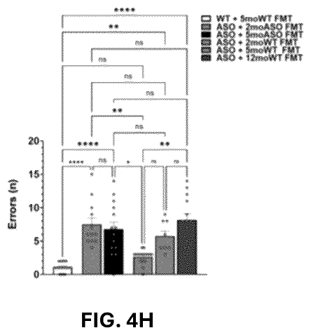

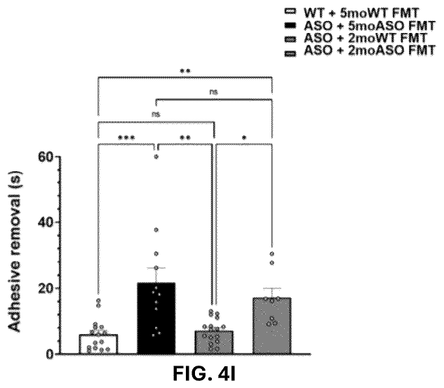

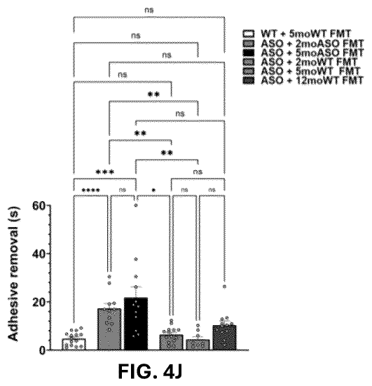

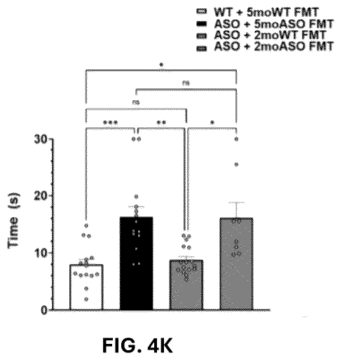

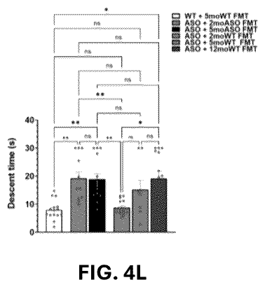

Next, the effect of FMT on improving motor symptoms in ASO animals was tested. For this analysis, FMT from 2 months old, 5 months old, and 12 months old WT mice and 2 months old and 5 months old ASO mice were transplanted into either 5-month-old WT or 5-month-old ASO mice. The weight of the tested animals was monitored throughout the process of the fecal transplant. FIGS. 4A-D illustrate the weight of the tested mice. Motor behavior metrics of tested mice as measured by monitoring beam transversal (FIGS. 4E-4H), adhesive removal (FIGS. 4I and 4J) and pole descend (FIGS. 4K and 4L) illustrate that fecal microbiome transplant from WT donors, specifically young WT donors improved motor symptoms like beam traversal, pole descend and adhesive removal in ASO recipients.

### Example 3. Fecal Microbiome Transplant from WT Donors Reduces αSyn Aggregation in ASO Recipients after 4 Weeks of Treatment

This example demonstrates that αSyn aggregation is reduced in ASO mice after FMT.

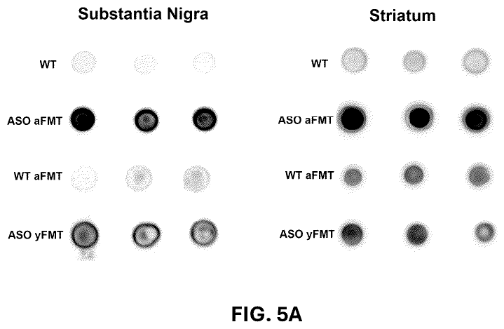

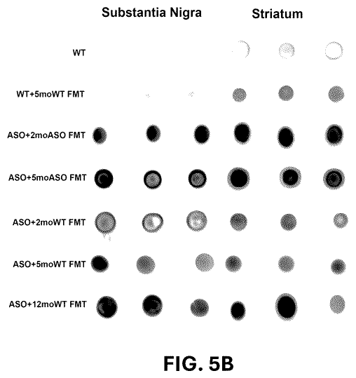

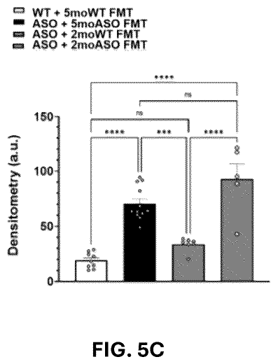

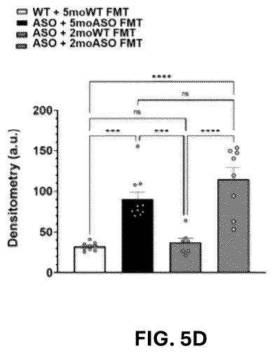

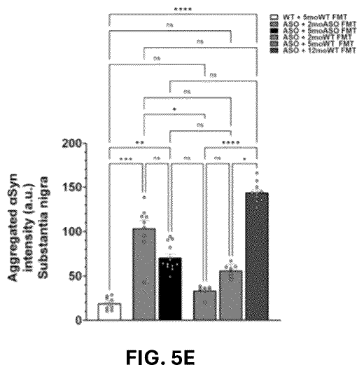

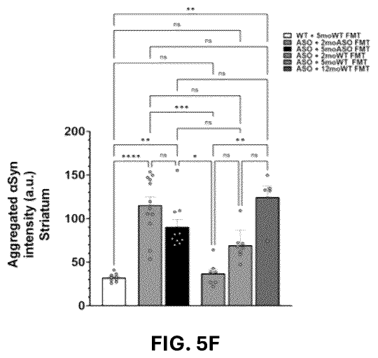

Motor deficits and GI symptoms in ASO mice coincide with the aggregation of αSyn. To test whether FMT has an effect on aggregation of αSyn, 5-month-old WT or 5-month-old ASO mice transplanted with FMT from 2 months old, 5 months old, and 12 months old WT mice and 2 months old and 5 months old ASO mice was tested for αSyn aggregation. As illustrated in FIGS. 5A and 5B, notable aggregation of αSyn in the substantia nigra (SN) and striatum brain regions was observed in ASO animals by dot blot. Surprisingly, ASO mice that received FMT from young WT mice (2-month-old or 5-month-old) displayed appreciably fewer αSyn aggregates (FIGS. 5A and 5B). Such an effect was not seen when the ASO mice received FMT from young ASO mice (2-month-old or 5-month-old). Altogether, these data indicate that the fecal microbiota reduces αSyn aggregation and/or prevent the clearance of insoluble protein aggregates. FIGS. 5C-5F show densitometry quantification of αSyn aggregation dot blots of substantia nigra (SN) and striatum brain regions.

Altogether, these data indicate that the fecal microbiota from WT donors, specifically young WT donors reduce αSyn aggregation and/or clearance of insoluble protein aggregates.

### Example 4. A Combination of Genotype and Microbiome is Necessary to Trigger Motor and Gastrointestinal Dysfunction

This example demonstrates that αSyn aggregation is reduced in ASO mice after FMT.

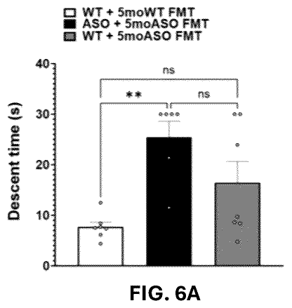

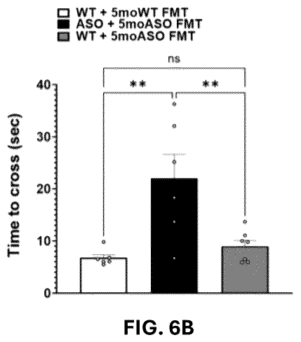

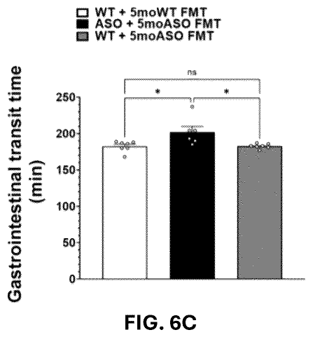

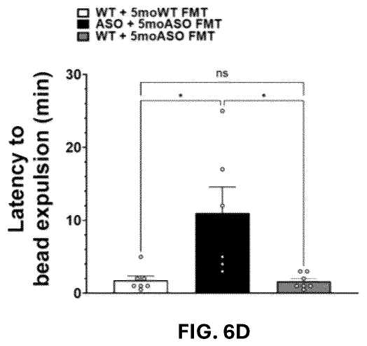

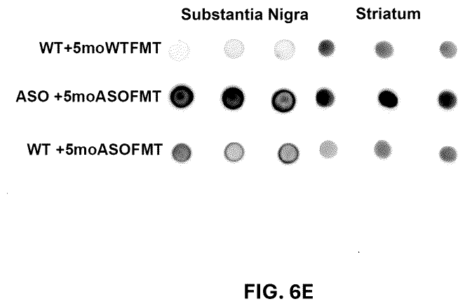

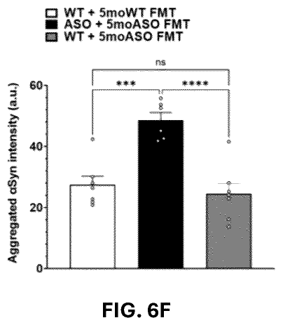

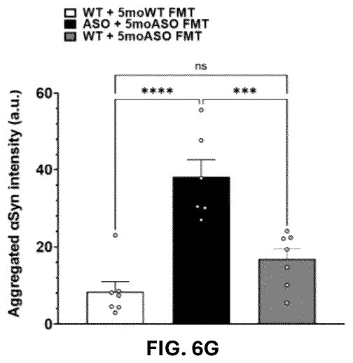

To further corroborate the effect of age and microbiome on motor and gastrointestinal dysfunction, 5-month-old WT and ASO mice were transplanted with fecal microbiome from 5-month-old WT and ASO mice and the effect of FMT on improving GI dysfunction, motor dysfunction and αSyn aggregation was tested. The motor functions as measured by descent time and time to cross (FIGS. 6A and 6B), gastrointestinal functions as measured by gastrointestinal transit time and latency bead expulsion (FIGS. 6C and 6D) and αSyn aggregation evaluated by dot-blots and densitometry quantification of αSyn aggregation dot blots of substantia nigra (SN) and striatum brain regions in these mice clearly indicate that fecal microbiota from young WT donors, but not young ASO mice showed an improved PD-related gastrointestinal and motor symptoms and reduced αSyn aggregation as illustrated in FIGS. 6E-6G.

### Example 5. Fecal Microbiome Transplant from Vancomycin-Treated 2-Month-Old Donor WT into 5-Month-Old ASO Animals does not Improve Motor or Gastrointestinal Function Symptoms

This example demonstrates that the effect of FMT on improving motor or gastrointestinal function symptoms is mediated through the fecal microbiome.

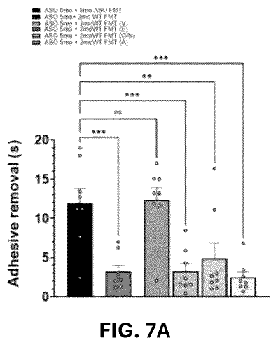

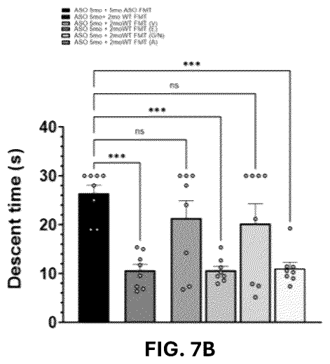

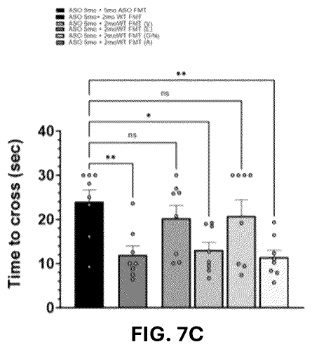

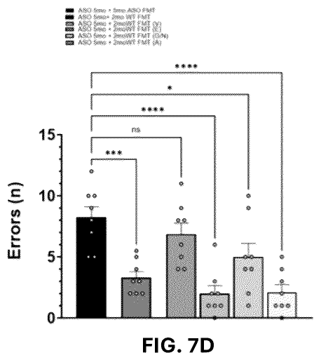

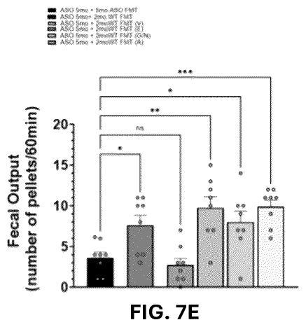

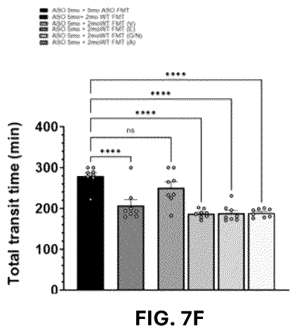

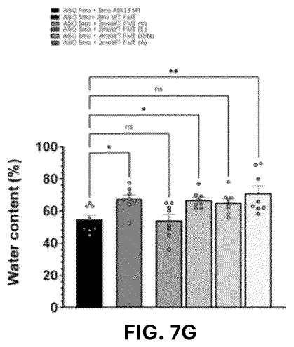

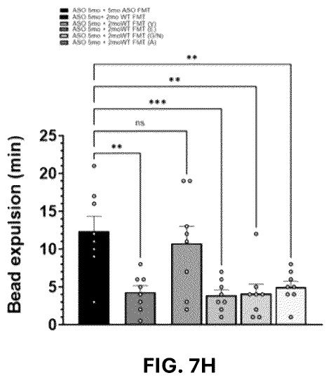

To test whether the effect of FMT observed in the above-mentioned examples was mediated by microbiome, 2-month-old WT donors were treated with individual antibiotics such as vancomycin (V), erythromycin (E), gentamycin/neomycin (G/N) or ampicillin (A) for 2 weeks. Following antibiotic treatment, the fecal microbiome from WT donors were transplanted into 5-month-old ASO mice previously treated with ABX cocktail as described herein and the effect of fecal microbiome on motor and gastrointestinal dysfunction and αSyn aggregation was evaluated. As shown in FIGS. 7A-7H, motor functions as measured by adhesive removal, (FIG. 7A) pole descent, (FIG. 7B) beam crossing (FIG. 7C) and number of errors on beam crossing (FIG. 7D), gastrointestinal functions as measured by fecal output (FIG. 7E), gastrointestinal transit time (FIG. 7F), water content (FIG. 7G) and latency bead expulsion (FIG. 7H) were improved or reduced in ASO mice treated with FMT from erythromycin, ampicillin, gentamycin/neomycin-treated WT mice but not with FMT from vancomycin-treated WT mice. This illustrates that the effect of FMT on improving motor or gastrointestinal function symptoms is mediated through the vancomycin-sensitive fecal microbiome.

### Example 6. Proteomic Analysis Following Fecal Transplant Upregulates Proteins Involved in Degradation by the Lysosome

This example demonstrates the proteomic analysis of the substantia nigra region of the brain of ASO animals after fecal transplant with 2-month-old WT fecal microbiota.

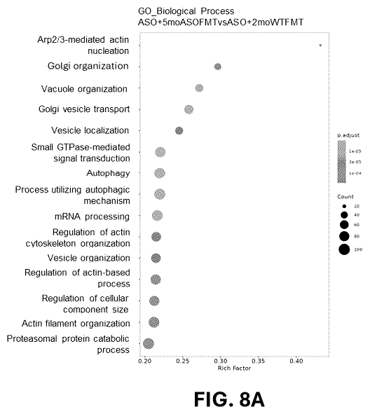

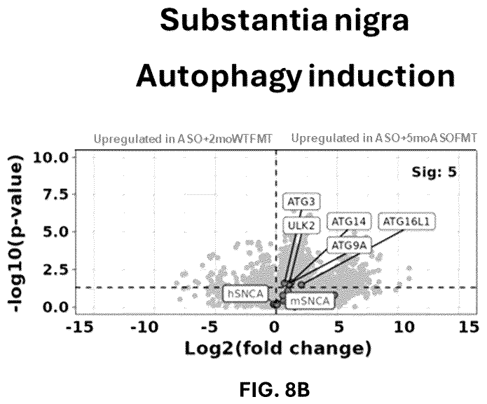

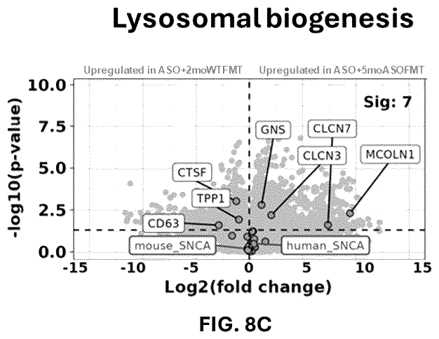

FIG. 8A illustrates the Gene ontology (GO) of the main biological process altered in the substantia nigra of ASO animals after fecal transplant with 2-month-old WT fecal microbiota. Proteins involved in regulating autophagy and lysosomal pathways was upregulated in ASO animals after fecal transplant with 2-month-old WT fecal microbiota as shown in FIGS. 8B and 8C.

### Example 7: Treatment of a Neurodevelopmental Disorder or a Symptom Thereof

This example illustrates the treatment of a patient suffering from a neurodevelopmental disorder or a symptom thereof. A human subject who exhibits neurodevelopmental disorder or a symptom thereof is identified. A composition comprising an effective amount of fecal microbiota from a healthy subject who is younger than the patient suffering from a neurodevelopmental disorder or a symptom thereof is administered to the patient. The administration of fecal microbiota is expected to alter the composition of gut bacteria in the patient. It is also expected that the fecal microbiota administration will improve neurodevelopmental disorder or a symptom thereof in the patient.

### Example 8: Treatment of a Parkinson's Disease or a Symptom Thereof

This example illustrates the treatment of a patient suffering from Parkinson's disease (PD) or a symptom thereof. A human subject who exhibits PD or a symptom thereof is identified. A composition comprising an effective amount of fecal microbiota from a healthy subject who is younger than the patient suffering from PD or a symptom thereof is administered to the patient. The administration of fecal microbiota is expected to alter the composition of gut bacteria in the patient. It is also expected that the fecal microbiota administration will improve PD or a symptom thereof in the patient.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A method of treating, improving and/or delaying a neurodegenerative disorder or a symptom thereof in a subject in need thereof, the method comprising:
identifying a subject having a neurodegenerative disorder, or a symptom thereof;
administering to the subject a composition comprising an effective amount of fecal microbiota,
wherein the fecal microbiota is derived from a healthy subject; and
wherein the neurodegenerative disorder or the symptom thereof of the subject is treated, improved and/or delayed after administering the composition.

2. The method of claim 1, wherein the healthy subject is younger as compared to the subject having the neurodegenerative disorder or the symptom thereof.

3. The method of claim 1, wherein the neurodegenerative disorder is a Parkinson's disease synucleinopathy or Huntington's disease.

4. The method of claim 1, wherein the subject has an abnormal level of aggregation of α-synuclein (αSyn).

5. The method of claim 1, wherein the method improves one or more physical impairments in the subject.

6. The method of claim 1, wherein the method improves one or more gastrointestinal (GI) functions of the subject.

7. The method of claim 6, wherein one or more GI functions comprise vomiting, dysphagia, bloating, sialorrhea, constipation, or combinations thereof.

8. The method of claim 1, wherein the method relieves constipation of the subject.

9. The method of claim 1, wherein the method relieves one or more motor deficit symptoms in the subject.

10. The method of claim 9, wherein the one or more motor deficit symptoms comprise tremors, muscle rigidity, bradykinesia, impaired gait, or any combination thereof.

11. The method of claim 1, wherein administering the composition decreases a rate and/or level of αSyn aggregation in a brain of the subject, and/or decreases a clearance rate and/or level of insoluble αSyn protein aggregate in the brain the subject, or a combination thereof.

12. The method of claim 1, wherein the subject suffers from a synucleinopathy.

13. The method of claim 12, wherein the synucleinopathy is Parkinson's disease, dementia with Lewy body disease, multiple system atrophy, or any combination thereof.

14. The method of claim 12, wherein the synucleinopathy is Parkinson's disease.

15. The method of claim 12, wherein the synucleinopathy is a primary or idiopathic parkinsonism, secondary or acquired parkinsonism, hereditary parkinsonism, Parkinson plus syndromes, multiple system degeneration, or any combination thereof.

16. A method of treating, improving and/or delaying a neurodegenerative disorder or a symptom thereof in a subject, the method comprising:
identifying a subject having the neurodegenerative disorder or a symptom thereof;
determining an age of the subject having the neurodegenerative disorder or a symptom thereof; and
administering to the subject a composition comprising an effective amount of fecal microbiota,
wherein the fecal microbiota is derived from a healthy subject, and
wherein the healthy subject is at least 10 years younger than the subject having the neurodegenerative disorder or a symptom thereof.

17. The method of claim 16, wherein the healthy subject is 20 years younger than the subject having the neurodegenerative disorder or a symptom thereof.

18. The method of claim 16, wherein the neurodegenerative disorder is a Parkinson's disease synucleinopathy or Huntington's disease.

19. The method of claim 16, wherein the subject in need has an abnormal level of aggregation of α-synuclein (αSyn).

20. The method of claim 16, wherein the method improves one or more gastrointestinal (GI) functions of the subject.

21. The method of claim 20, wherein one or more GI functions comprise vomiting, dysphagia, bloating, sialorrhea, constipation or combinations thereof.

22. The method of claim 16, wherein the method relieves constipation of the subject.

23. The method of claim 16, wherein the method relieves one or more motor deficit symptoms in the subject.

24. The method of claim 23, wherein the one or more motor deficit symptoms comprise tremors, muscle rigidity, bradykinesia, impaired gait, or any combination thereof.

25. The method of claim 1, wherein the composition is formulated for oral administration.

26. The method of claim 1, wherein the composition is formulated for intragastric administration.

27. The method of claim 1, wherein the healthy subject is not exposed to antibiotics prior to fecal microbiota collection.

28. The method of claim 1, wherein the composition comprising an effective amount of fecal microbiota is administered more than once to the subject.

29. The method of claim 1, wherein the composition comprising an effective amount of fecal microbiota is administered every day, monthly or weekly.

30. The method of claim 1, wherein the subject or the subject in need is not receiving an antibiotic treatment.

31. The method of claim 1, wherein the composition is a probiotic composition, a nutraceutical composition, a pharmaceutical composition, or any combination thereof.
